(19) United States
(12) Patent Application Publication
(10) Pub. No.: US 2010/0130744 A1
(43) Pub. Date: May 27, 2010

(54) PROCESS FOR THE PREPARATION OF ARIPIPRAZOLE

(76) Inventors: Mahesh Nagarimadugu, Andhra Pradesh (IN); Vipin Kumar Kaushik, Andhra Pradesh (IN); Ramesh Dandala, Andhra Pradesh (IN); Sivakumaran Meenakshisunderam, Andhra Pradesh (IN)

Correspondence Address: Jay R Akhave;Patent Science, 2058 N Mills Ave #612, Claremont, CA, 91711, US

(21) Appl. No.: 12/451,775

(22) PCT Filed: May 29, 2008
(86) PCT No.: PCT/IB2008/001446

§ 371 Date: Nov. 30, 2009

(30) Foreign Application Priority Data
Jun. 1, 2007 (IN) 1141/CHE/2007

(51) Int. Cl. C07D 401/12

(52) U.S. Cl. 544/363

(57) ABSTRACT

The present invention relates to an improved process for the preparation of 7-[4-[4-(2,3-dichlorophenyl)-1-piper-azinyl]butoxy]-3,4-dihydro-2(1H)-quinolinone of Formula (I).

### FIELD OF THE INVENTION

The present invention relates to an improved process for the preparation of 7-[4-[4-(2,3-dichlorophenyl)-1-piperazinyl]butoxy]-3,4-dihydro-2(1H)-quinolinone of Formula (I).

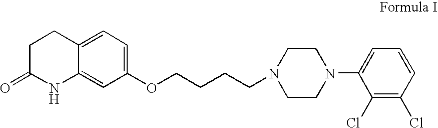

### BACKGROUND OF THE INVENTION

7-[4-[4-(2,3-Dichlorophenyl)-1-piperazinyl]butoxy]-3,4-dihydro-2(1H)-quinolinone, generically known as Aripiprazole, is psychotherapic drug. Aripiprazole is approved specifically for the treatment of schizophrenia. The activity of Aripiprazole is proposed to be through mediation of combination of partial agonist activity of D₂ and 5-HT_{1A} receptors and antagonist activity at 5-HT_{2A} receptors. Aripiprazole is marketed as oral tablets under the trade name of Abilify®.

Otsuka Pharmaceutical Co., Ltd. has generically disclosed Aripiprazole in U.S. Pat. No. 4,734,416, subsequently, Aripiprazole has been specifically disclosed in U.S. Pat. No. 5,006,528.

U.S. Pat. No. 5,006,528, discloses a process for the preparation of Aripiprazole, which comprises alkylating the hydroxy group of 7-hydroxy-3,4-dihydro-2(1H)-quinolinone of Formula (II) with 1,4-dibromobutane of Formula (III) in presence of potassium carbonate in water to produce 7-(4-bromobutoxy)-3,4-dihydro-2(1H)-quinolinone of Formula (IV), which is purified by column chromatography using dichloromethane as an eluent and recrystallised from a mixture of n-hexane and ethanol. 7-(4-Bromobutoxy)-3,4-dihydro-2(1H)-quinolinone (IV) is further condensed with 1-(2,3-dichlorophenyl)piperazine (V) in presence of sodium iodide and acetonitrile to obtain Aripiprazole.

The process is as shown in Scheme-I below:

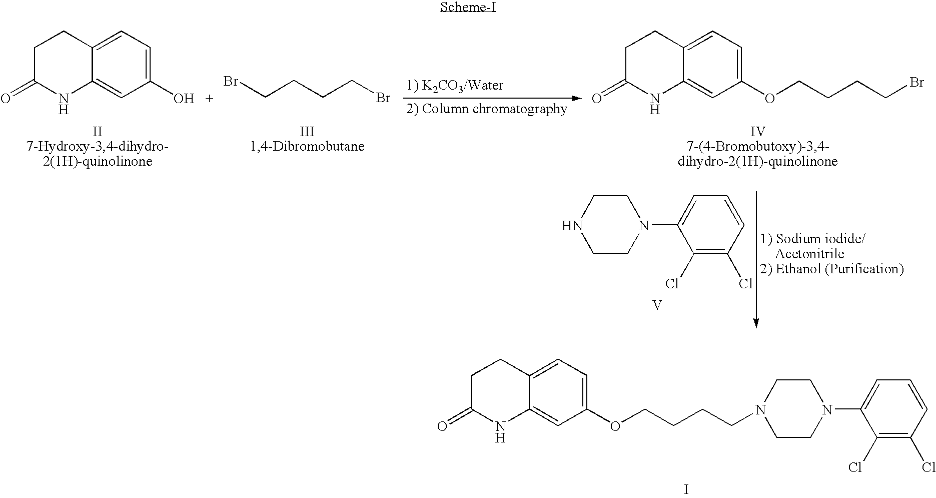

Journal of Medicinal Chemistry, Vol. 41, No. 5, 658-667, (1988) discloses a process for the preparation of 7-(4-bromobutoxy)-3,4-dihydro-2(1H)-quinolinone (IV) by alkylation of 3,4-dihydro-7-hydroxy-2(1H)-quinolinone (II) with 1,4-dibromobutane (III) in the presence of potassium carbonate in N,N-dimethylformamide (DMF).

US 2005/0215585 A1 discloses a process for the preparation of 7-(4-bromobutoxy)-3,4-dihydro-2(1H)-quinolinone (IV) by reacting 3,4-dihydro-7-hydroxy-2(1H)quinolinone (II) with 1,4-dibromobutane (III) in presence of base under neat conditions.

7-(4-Bromobutoxy)-3,4-dihydro-2(1H)-quinolinone (IV) obtained by the above prior-art methods contained around 10% of unwanted dimer 1,4-bis[3,4-dihydro-2(1H)quinolinone-7-oxy]butane of Formula (VI) as an impurity, which causes low yield and low purity of finished product, Aripiprazole.

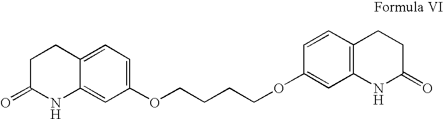

1,4-Bis[3,4-dihydro-2(1H)quinolinone-7-oxy]butane (VI) cannot be removed by crystallization and the only way to remove the impurity is by column chromatography. Employing column chromatography technique is tedious and laborious and also involves use of large quantities of solvents, and hence is not suitable for industrial scale operations.

Hence, there is a need to develop a process, which provides 7-(4-bromobutoxy)-3,4-dihydro-2(1H)-quinolinone (IV) with high purity specifically with less content of 1,4-bis[3,4-dihydro-2(1H)-quinolinone-7-oxy]butane (VI).

The present invention is specifically directed towards the purification of 7-(4-bromobutoxy)-3,4-dihydro-2(1H)-quinolinone (IV) which reduces the unwanted dimer impurity to a pharmaceutically acceptable limit, which inturn provides Aripiprazole of high purity and improved yield.

### OBJECTIVE OF THE INVENTION

The main objective of the present invention is to provide a simple and effective process for the preparation of Aripiprazole with high purity and good yields on a commercial scale.

### SUMMARY OF THE INVENTION

Accordingly, the present invention provides a process for the preparation of 7-[4-[4-(2,3-dichlorophenyl)-1-piperazinyl]butoxy]-3,4-dihydro-2(1H)-quinolinone (Aripiprazole) of Formula (I) through an intermediate 7-(4-bromobutoxy)-3,4-dihydro-2(1H)-quinolinone, having dimer impurity (VI) less than 0.5%, which comprises;
(i) reacting 7-hydroxy-3,4-dihydro-2(1H)-quinolinone of Formula (II)

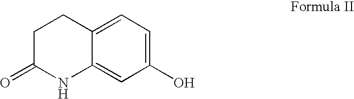

with 1,4-dibromobutane (III) in presence of a base and solvent to produce 7-(4-bromobutoxy)-3,4-dihydro-2(1H)-quinolinone of Formula (IV)

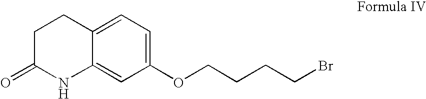

(ii) treating the compound of Formula (IV) with silica gel in a solvent to produce pure 7-(4-bromobutoxy)-3,4-dihydro-2(1H)-quinolinone of Formula (IV) having dimer impurity 1,4-bis[3,4-dihydro-2(1H)quinolinone-7-oxy]butane of Formula (VI) less than about 0.5%

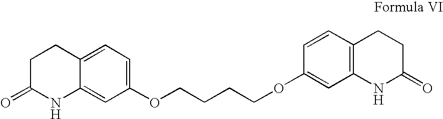

(iii) reacting pure compound of Formula (IV) with 1-(2,3-dichlorophenyl)piperazine of Formula (V) or its salt

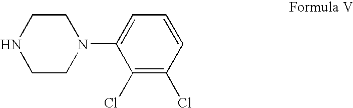

in presence of base and alkali iodide in a solvent to produce 7-[4-[4-(2,3-dichlorophenyl)-1-piperazinyl]butoxy]-3,4-dihydro-2(1H)-quinolinone of Formula (I).

### DETAILED DESCRIPTION OF THE INVENTION

The present invention relates to an improved process for the preparation of 7-[4-[4-(2,3-dichlorophenyl)-1-piperazinyl]butoxy]-3,4-dihydro-2(1H)-quinolinone (Aripiprazole) of Formula (I).

7-Hydroxy-3,4-dihydro-2(1H)-quinolinone of Formula (II) is reacted with 1,4-dibromobutane of Formula (III) in presence of base selected from sodium carbonate, potassium carbonate, calcium carbonate, cesium carbonate, sodium bicarbonate, sodium hydroxide, potassium hydroxide, calcium hydroxide or mixtures thereof in a solvent selected from water, ethers such as dioxane, tetrahydrofuran, ethylene glycol dimethyl ether and the like or mixture thereof; aromatic hydrocarbons such as toluene, xylene and the like or mixture thereof; lower alcohols such as methanol, ethanol, isopropanol and the like or mixture thereof; polar solvents such as dimethylformamide (DMF), dimethyl sulfoxide (DMSO), acetonitrile, dimethylacetamide and the like or mixture thereof. The reaction is carried out at reflux temperature. After completion of reaction, reaction mass is cooled to room temperature and the organic layer is separated. The organic layer containing 7-(4-bromobutoxy)-3,4-dihydro-2(1H)-quinolinone of Formula (IV) having 5 to 10% of dimer impurity 1,4-bis[3,4-dihydro-2(1H)quinolinone-7-oxy]butane of Formula (VI) is washed with pre-cooled aqueous base selected from sodium hydroxide, potassium hydroxide, sodium carbonate or potassium carbonate and the resulting organic layer is concentrated under reduced pressure. The obtained residue is diluted with organic solvent selected from toluene, methylene chloride, ethyl acetate, diethylether, xylene, methyl ethyl ketone and treated with silica gel at 55-60° C. and the resulting reaction mass is stirred for ½ hr to 2 hrs at 60-70° C. Silica gel is removed by filtration and the resulting filtrate is concentrated to residue under reduced pressure to obtain pure 7-(4-bromobutoxy)-3,4-dihydro-2(1H)-quinolinone (IV) as a residue; which is further crystallised by using solvent system selected from hexanes, cyclohexane, heptane, and methanol, ethanol. isopropanol, butanol or mixtures thereof.

7(4-Bromobutoxy)-3,4-dihydro-2(1H)-quinolinone (IV) produced by the above purification process results in dimer impurity (VI) to less than 0.5% by HPLC analysis. The major advantage realized with the process of the present invention is that the removal of dimer impurity without the use of tedious techniques such as column chromatography, conventional distillation techniques. The present technique is very easy and reduces the dimer impurity, which otherwise cannot be reduced crystallization techniques.

7-(4-Bromobutoxy)-3,4-dihydro-2(1H)-quinolinone (IV) is reacted with 1(2,3-dichlorophenyl)piperazine hydrochloride of Formula (V) in presence of base selected from sodium carbonate, potassium carbonate, calcium carbonate or cesium carbonate or mixtures thereof and alkali iodide selected from sodium iodide, potassium iodide, calcium iodide, in a solvent selected from dimethylformamide (DMF), dimethyl sulfoxide (DMSO), acetonitrile, dimethylacetamide and the like or mixture thereof. The reaction is carried out at a temperature of about 75 to about 100° C. After completion of reaction, the reaction mass is cooled to a temperature of about 40 to 55° C. and filtered to remove insoluble material. DM water is added, to the resulting filtrate and the temperature of the resulting suspension is raised to about 100° C. to obtain a clear solution, which is cooled to 25-30° C. and stir for ½ hr to about 1 hr, and filter the precipitated crude Aripiprazole. Crude Aripiprazole is recrystallised from ethanol, methanol.

The details of the process of the invention are provided in the examples given below, which are provided by way of illustration only and therefore should not be construed to limit the scope of the invention.

### Example-1

### Step A:

### Preparation of Pure 7-(4-Bromobutoxy)-3,4-Dihydro-2(1H)-Quinolinone (IV):

7-Hydroxy-3,4-dihydro-2(1H)-quinolinone (20 g, 0.153 mol) was added to a solution of potassium carbonate (25 g, 0.181 mol) in DM water (400 ml) at 25-35° C. and the contents were heated to 80-90° C. to obtain a clear solution. 1,4-Dibromobutane (200 ml) was added to the reaction mass, heated to reflux temperature (100-105° C.) and stirred for 7 hours at the same temperature. After completion of reaction, reaction mass was cooled to ambient temperature and organic layer was separated. Organic layer containing 7-(4-bromobutoxy)-3,4-dihydro-2(1H)-quinolinone having 5% 1,4-bis[3,4-dihydro-2(1H)quinolinon-7-oxy]butane (by HPLC, by area normalization) was washed with precooled 5% w/v aqueous sodium hydroxide (75 ml) at 15-20° C., to remove unreacted 7-hydroxy-3,4-dihydro-2(1H)-quinolinone. Organic layer was concentrated at 90-105° C. under reduced pressure varying from 50 to 5 mm of Hg to dryness. The concentrated mass was diluted with toluene (2000 ml) and heated to 60° C. Silica gel (50 g) was added and stirred for 30 min at 60-70° C. Silica gel was removed by filtration and the filtrate was concentrated at 60-80° C. under reduced pressure varying from 200 to 10 mm of Hg. The resulting concentrated mass having 0.3% of 1,4-bis[3,4-dihydro-2(1H)quinolinon-7-oxy]butane (by HPLC, by area normalization) was diluted with hexanes (50 ml) and stirred for 30 minutes to crystallize the product. Product was filtered and washed with hexanes.

Yield: 18 g

Chromatographic purity: 97.89% (by HPLC, by area normalization)

1,4-Bis[3,4-dihydro-2(1H)quinolinone-7-oxy]butane content: 0.33%

Step B:

Preparation of 7-[4-[4-(2,3-Dichlorophenyl)-1-Piperazinyl]Butoxy]-3,4-Dihydro-2(1H)-Quinolinone (I) (Aripiprazole):

A suspension of 7-(4-bromobutoxy)-3,4-dihydro-2(1H)-quinolinone (10 g, 0.034 mol), sodium iodide (7.9 g, 0.052 mol), sodium carbonate (7.8 g, 0.073 mol), 1-(2,3-dichlorophenyl)piperazine hydrochloride (9.7 g, 0.036 mol) in N,N-dimethylformamide (80 ml) was stirred 90-100° C. The reaction, was monitored by qualitative HPLC. After completion of reaction, the reaction was cooled to 50-55° C. and undissolved matter was removed by filtration. DM water (20 ml) was added to obtained filtrate and temperature of the resulting suspension was raised to 100° C. to obtain a clear solution. Resultant clear solution was cooled to 25-30° C., stirred for 30 minutes and filtered. The solid, thus obtained was recrystallized from ethanol (absolute alcohol) to yield 11.5 g of Aripiprazole with 99.93% purity by HPLC.

### Example-2

### Preparation of Pure 7-(4-Bromobutoxy)-3,4-Dihydroquinolinone

7-Hydroxy-3,4-dihydroquinolinone (20 g, 0.153 mol) was added to a solution of potassium carbonate (25 g, 0.181 mol) in DM water (140 ml) at 25-35° C. and the contents were heated to 65-70° C. to obtain a clear solution. 1,4-Dibromobutane (200 ml) was added to the reaction mass and again heated to reflux temperature (95-100° C.) and stirred for 5 hours at the same temperature. After completion of reaction, reaction mass was cooled to ambient temperature and organic layer was separated. Organic layer containing 1,4-bis[3,4-dihydro-2(1H)quinolinon-7-oxy]butane was washed with precooled 5% w/v aqueous sodium hydroxide (75 ml) at 10-15° C., to remove unreacted 7-hydroxy-3,4-dihydroquinolinone. Organic layer was concentrated at 100-120° C. under reduced pressure varying from 50 to 5 mm of Hg. The concentrated mass was diluted with toluene (500 ml) and heated to 60° C. Silica gel (60 g) was added and stirred for 30 min at 60-70° C. Silica gel was removed by filtration and treated again with preheated toluene (1×400 ml, 1×200 ml, 60-70° C.). The combined filtrate was concentrated at 60-70° C. under reduced pressure varying from 200 to 10 mm of Hg. Thus, obtained concentrated mass was diluted with hexanes (150 ml) and stirred for 30 minutes upon which the product crystallized out. Product was filtered and washed with hexanes. Yield: 17 g

Chromatographic purity: 97.31% (by HPLC, by area normalization)

1,4-Bis[3,4-dihydro-2(1H)quinolinon-7-oxy]butane content: 0.44%

### Example-3

### Preparation of Pure 7-(4-Bromobutoxy)-3,4-Dihydroquinolinone

7-Hydroxy-3,4-dihydroquinolinone (50 g, 0.306 mol) was added to a solution of potassium carbonate (62.5 g, 0.453 mol) in DM water (350 ml) at 25-35° C. and the contents were heated to 65-70° C. to obtain a clear solution. 1,4-Dibromobutane (500 ml) was added to the reaction mass and again heated to reflux temperature (95-100° C.) and stirred for 5 hours at the same temperature. After completion of reaction, reaction mass was cooled to ambient temperature and organic layer was separated. Organic layer containing 1,4-bis[3,4-dihydro-2(1H)quinolinon-7-oxy]butane was washed with precooled 5% w/v aqueous sodium hydroxide (195 ml) at 10-15° C., to remove unreacted 7-hydroxy-3,4-dihydroquinolinone. Organic layer was concentrated at 100-120° C. under reduced pressure varying from 50 to 5 mm of Hg. The concentrated mass was diluted with toluene (1000 ml) and heated to 60° C. Silica gel (165 g) was added and stirred for 30 min at 60-70° C. Silica gel was removed by filtration and treated again with preheated toluene (2×750 ml, 60-70° C.). The combined filtrate was concentrated at 60-70° C. under reduced pressure varying from 200 to 10 mm of Hg. Thus, obtained concentrated mass was diluted with cyclohexane (150 ml) and stirred for 30 minutes to crystallize the product. Product was filtered and washed with cyclohexane. Yield: 42.5 g

Chromatographic purity: 97.5% (by HPLC, by area normalization)

1,4-Bis[3,4-dihydro-2(1H)quinolinon-7-oxy]butane content: 0.55%

### Example-4

### Preparation of Pure 7-(4-Bromobutoxy)-3,4-Dihydroquinolinone

7-Hydroxy-3,4-dihydroquinolinone (30 g, 0.184 mol) was added to a solution of potassium carbonate (37 g, 0.268 mol) in DM water (1200 ml) at 25-35° C. and the contents were heated to 65-70° C. to obtain a clear solution. 1,4-Dibromobutane (150 ml) was added to the reaction mass and again heated to reflux temperature (95-100° C.) and stirred for 5 hours at the same temperature. After completion of reaction, reaction mass was cooled to ambient temperature and organic layer was separated. Organic layer containing 1,4-bis[3,4-dihydro-2(1H)quinolinon-7-oxy]butane was washed with precooled 5% w/v aqueous sodium hydroxide (110 ml) at 10-15° C., to remove unreacted 7-hydroxy-3,4-dihydroquinolinone. Organic layer was concentrated at 100-120° C. under reduced pressure varying from 50 to 5 mm of Hg. The concentrated mass was diluted with xylene (750 ml) and heated to 60° C. Silica gel (120 g) was added and stirred for 30 min at 60-70° C. Silica gel was removed by filtration and treated again with preheated xylene (2×750 ml, 60-70° C.). The combined filtrate was concentrated at 60-70° C. under reduced pressure varying from 200 to 10 mm of Hg. Thus, obtained concentrated mass was diluted with cyclohexane (150 ml) and stirred for 30 minutes. Product was filtered and washed with cyclohexane. Yield: 25 g

Chromatographic purity: 97.24% (by HPLC, by area normalization)

1,4-Bis[3,4-dihydro-2(1H)quinolinon-7-oxy]butane content: 0.29%

### Example-5

### Preparation of 7-[4-[4-(2,3-Dichlorophenyl)-1-Piperazinyl]Butoxy]-3,4-Dihydro-2(1H)-Quinolinone (I) (Aripiprazole)

A suspension of 7-(4-bromobutoxy)-3,4-dihydro-2(1H)-quinolinone (120 g, 0.40 mol), sodium iodide (12 g, 0.08 mol), sodium carbonate (85.35 g, 0.82 mol) and 1-(2,3-dichlorophenyl)piperazine hydrochloride (116.4 g, 0.44 mol) in N,N-dimethylformamide (540 ml) was stirred at 93-98° C. The reaction was monitored by qualitative HPLC. After completion of reaction, the reaction mass was cooled to 60° C., and un-dissolved matter was removed by filtration. The obtained filtrate was cooled to 8-10° C., stirred for 45 min, filtered and washed with N,N-dimethylformamide followed by DM water. The solid, thus obtained was dried to constant weight. Aripiprazole (121 g) having chromatographic purity of 99.49% and 1,4-bis(3,4-dihydro-2(1H)quinolinon-7-oxy) butane (0.12%).

### Example-6

### Purification of 7-[4-[4-(2,3-Dichlorophenyl)-1-Piperazinyl]Butoxy]3,4-Dihydro-2(1H)-Quinolinone (I) (Aripiprazole)

A suspension of Aripiprazole (100 g) in 2300 ml 20% w/v aqueous ethanol was heated to 78-80° C. to obtain a clear solution. The obtained solution was treated with carbon and filtered at 78-80° C. The filtrate, thus obtained was slowly cooled to 8-10° C., and stirred for 45 min and filtered. Aripiprazole hydrate, thus obtained was dried at 76-80° C. to yield 89 g of Aripiprazole crystalline Type-I (as reported in ‘The Fourth Japan-Korea Symposium on Separation Technology’, 1996, 937-940) having chromatographic purity 99.97% and 1,4-bis(3,4-dihydro-2(1H)quinolinon-7-oxy)butane ‘Not detected’.

We claim:

1. An Improved process for the preparation of 7-[4-[4-(2,3-dichlorophenyl)-1-piperazinyl]butoxy]-3,4-dihydro-2(1H)-quinolinone (Aripiprazole) of Formula (I) 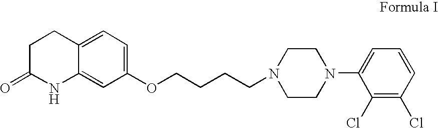
which comprises,
(i) reacting 7-hydroxy-3,4-dihydro-2(1H)-quinolinone of Formula (II) 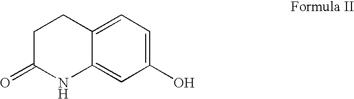
with 1,4-dibromobutane (III) in presence of base and solvent to produce 7-(4-bromobutoxy)-3,4-dihydro-2(1H)-quinolinone of Formula (IV) 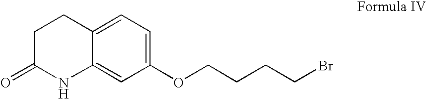
(ii) treating the compound of Formula (IV) with silica gel in a solvent to produce pure 7-(4-bromobutoxy)-3,4-dihydro-2(1H)-quinolinone of Formula (IV) having dimer impurity 1,4-bis[3,4-dihydro-2(1H)quinolinone-7-oxy]butane of Formula (VI) less than about 0.5% 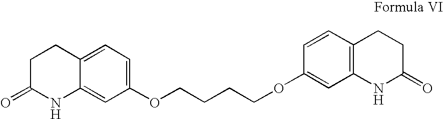
(iii) reacting pure compound of Formula (IV) with 1-(2,3-dichlorophenyl)piperazine of Formula (V) or its salt 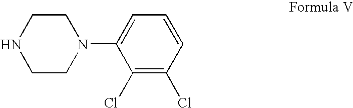
in presence of base and alkali iodide in a solvent to produce 7-[4-[4-(2,3-dichlorophenyl)-1-piperazinyl]butoxy]-3,4-dihydro-2(1H)-quinolinone of Formula (I).

2. A process according to claim 1, wherein the base used in step (i) is selected from sodium carbonate, potassium carbonate, calcium carbonate, cesium carbonate, sodium bicarbonate, sodium hydroxide, potassium hydroxide, calcium hydroxide or mixtures thereof.

3. A process according to claim 1, wherein the solvent used in step (i) is selected from water, ethers such as dioxane, tetrahydrofuran, ethylene glycol dimethyl ether and the like or mixture thereof; aromatic hydrocarbons such as toluene, xylene and the like or mixture thereof; lower alcohols such as methanol, ethanol, isopropanol and the like or mixture thereof; polar solvents such as dimethylformamide (DMF), dimethyl sulfoxide (DMSO), acetonitrile, dimethylacetamide and the like or mixture thereof.

4. A process according to claim 1, wherein the solvent used in step (ii) is selected from toluene, methylene chloride, ethyl acetate, diethyl ether, xylene, methyl ethyl ketone.

5. A process according to claim 1, wherein the base used in step (iii) is selected from sodium carbonate, potassium carbonate, calcium carbonate or cesium carbonate or mixture thereof.

6. A process according to claim 1, wherein the alkali iodide used in step (iii) is selected from sodium iodide, potassium iodide, calcium iodide or mixture thereof.

7. A process according to claim 1, wherein the solvent used in step (iii) is selected from dimethylformamide (DMF), dimethylsulfoxide (DMSO), acetonitrile, dimethylacetamide and the like or mixture thereof.
